(19) United States
(12) Patent Application Publication
(10) Pub. No.: US 2009/0306119 A1
(43) Pub. Date: Dec. 10, 2009

(54) Non-Codeine Opioid Analgesic Process And Formulations

(76) Inventor: Michael Keane, Victoria (AU)

Correspondence Address: Peter G Carroll;Medlen & Carroll, 101 Howard Street, Suite 350, San Francisco, CA, 94105, US

(21) Appl. No.: 12/225,035

(22) PCT Filed: Mar. 15, 2007
(86) PCT No.: PCT/AU2007/000324

§ 371 Date: Mar. 12, 2009

(30) Foreign Application Priority Data
Mar. 15, 2006 (AU) 2006901340

(51) Int. Cl. A61K 31/485; A61P 25/04

(52) U.S. Cl. 514/282

(57) ABSTRACT

The invention is directed to the relief of pain using opioid analgesics other than codeine, preferably in combination with a non-steroidal anti-inflammatory agent. Treatment is by self-administration. Compositions for the treatments are provided. Compositions of opioid analgesics other than codeine adapted to avoid the problems of potential abuse are also provided. The compositions have potential use as both prescription and non-prescription medications.

### FIELD OF THE INVENTION

The invention relates to self-medication and to an analgesic composition that is formulated so as to be able to be provided over-the-counter, as a prescription medicine or otherwise with less regulation as to supply, to an individual requiring analgesia or otherwise suffering from pain.

### BACKGROUND OF THE INVENTION

In most countries access to pharmaceuticals is subject to varying levels of control depending on the type of active ingredient in the pharmaceutical substance. Generally speaking, three levels of control can be recognised and these can be seen to broadly define three classes of medicine. In this specification, these will be referred to as over-the-counter medicines, prescription medicines and highly controlled medicines.

Over-the-counter (OTC) medicines are pharmaceuticals that a consumer may obtain from a pharmacy, supermarket or like outlet without being required to present a prescription or other authorisation from a medical practitioner or like health care service provider. As such, OTC medicines are primarily used to treat conditions that do not require a form of intervention by a medical practitioner or like health care service provider such as examination, diagnosis, consultation or advice. OTC medicines typically must be proven to be reasonably safe, generally well-tolerated and possess little or no abuse potential. In Australia, these medicines are currently evaluated by the Over-The-Counter Medicines Section of the Therapeutic Goods Administration of Australia (TGA). In the US they are evaluated by the Centre For Drug Evaluation and Research of the FDA (CDER). In the UK they are evaluated by Medicine and Healthcare products Regulatory Agency (MHRA).

OTC medicines are an important element of a health care system as they allow a consumer to self-medicate. Self-medication is a process for the treatment of a condition by the individual that suffers from it. It is generally characterised by self diagnosis (i.e. the individual with the condition diagnosing him/herself), decision making by the individual with the condition as to whether he/she should administer to him/herself a medicine and optionally the individual with the condition administering the medicine to him/herself. All of this generally occurs without the intervention by a health care service provider and the medicine is generally an OTC medicine, i.e. one obtained without prescription from a health care provider. The practice of self-medication in society helps to minimise the burden on healthcare services and allows individuals to obtain medication in circumstances when visiting a medical practitioner is difficult, such as after normal office hours.

In contrast, prescription medicines are those medicines that are regulated so as to require a prescription or other authorisation from a medical practitioner before a consumer can obtain access to the medicine. Prescription medicines may be pharmaceuticals containing higher dosages of active ingredient than equivalent OTC medicines. For example, codeine containing pharmaceuticals are available as prescription medicines at higher doses than their OTC counterparts. Prescription medicines may also be used to treat conditions that require initial medical diagnosis or minimal medical supervision or for active ingredients that a consumer may build up a resistance to, such as antibiotics.

Prescription medicines also play an important role in a health care system because they provide for access to more potent or specialised pharmaceuticals and treatment of more serious medical conditions under at least the initial supervision of a medical practitioner, while allowing a consumer to remain in his or her normal home environment. A consumer, after consulting with a medical practitioner is able to obtain the prescription medicine and then administer it to themselves in accordance with the instructions given by the medical practitioner, typically in the prescription. All this can be done without need for triaging.

A third level of regulation of pharmaceuticals is the highly controlled medicines. In addition to a prescription from a medical practitioner, there are various requirements for supply to patients. These include, for example, triaging, extra documentation, supervision and oversight by a medical practitioner and restriction on the type of medical practitioner that can authorise prescriptions. In these instances, the medical practitioner closely controls the administration of the pharmaceutical to the consumer. For highly controlled medicines, the extra layers of regulation reduce the timely availability of these medicines to treat conditions. Pharmaceuticals that have the potential for severe adverse side effects if administered incorrectly or abused by a consumer are typically regulated in this category.

In a health care system, highly controlled medicines are important as consumers with serious or life threatening conditions are able to obtain access to stronger medicines, while the potential for fatalities or complications resulting from incorrect administration, abuse or misuse are minimised due to the high level of control by medical practitioners.

Different types of analgesics are available as OTC medicines, prescription medicines and highly controlled medicines. A number of analgesic compositions containing codeine have been registered by the Therapeutic Goods Administration of Australia, and similar regulatory agencies in other countries, to be provided as OTC medicines and prescription medicines. These compositions may also contain a further analgesic compound, such as paracetamol or ibuprofen. These codeine containing OTC medicines are used by consumers to treat pain, such as headaches, backache and toothache, by self-medication.

One problem with codeine containing medicines is that they do not provide analgesia to all consumers as some consumers cannot effectively metabolise the active ingredient. Specifically, about 10% of the population do not have any or sufficient cytochrome P450 2D6 activity. This enzyme is required to convert codeine to morphine, the latter providing the analgesic activity. Other individuals that do not obtain analgesia from codeine containing medicines are those being treated with 2D6 inhibitors, such as fluoxetine.

There is a further sub-population that it would be unpredictable and potentially dangerous to treat with analgesic compositions containing codeine as, in contrast, they convert an abnormally large amount of morphine from codeine. Hence the administration of what is otherwise considered in the context of the general population to be a therapeutic dose of codeine puts these individuals in danger of overdose.

Further, there is generally a poor correlation between the amount of codeine administered and therapy obtained from individual to individual.

To summarise, there is a significant class of individuals that are unable to effectively or safely use codeine containing medicines. For these individuals, no effective OTC or prescription medicine is available.

There are numerous options available for developing new analgesic compositions which may be suitable for self-medication and OTC dispensing or dispensing with less regulation generally. For instance, known pharmaceuticals may be screened for a secondary medical indication as an analgesic. Alternatively, new biologically active compounds that provide analgesia may be identified. Such drug development and screening processes are time consuming and costly activities.

Although opioids provide strong analgesia, at the time of the invention herein, the consensus has generally been that it is not appropriate to make these compounds available as OTC or prescription medicines. One particular concern has been the belief that these compounds are highly addictive—more so than codeine—and hence are associated with a greater potential for abuse. Further these compounds can cause respiratory depression and death. The UN Single Convention for Narcotic Drugs, 1961 was ratified by many countries in recognition of these concerns.

The provision of pain relief is an important aspect of health care at all levels of regulation of medicines. Pain relief may involve the complete abatement of pain or merely the reduction of pain to a more tolerable level, for instance a level at which a person can continue to undertake their everyday tasks.

There is a need for new OTC medicines for providing analgesia or relief from pain.

There is also a need for new medicines which are subject to less regulation as to their supply for providing analgesia or relief from pain.

### SUMMARY OF THE INVENTION

The inventor has now determined that, contrary to generally accepted opinion, opioids, particularly morphine, hydromorphone and oxycodone, when provided in a suitable form, do not present a significantly different or increased abuse potential in the population as a whole compared to the abuse potential for codeine. Therefore, the inventor has determined that the perceived risks of unlimited abuse of, and dependence on, opioids other than codeine if available to consumers for self-medication are unfounded. Consequently, the inventor has discovered that opioids may be formulated and sold as OTC and/or prescription medicines.

Further, such compounds are capable of providing improved analgesia for the population as a whole. This is because opioids other than codeine provide a more widely acceptable analgesic option, which is not subject to the disadvantage of under- or over-metabolism of codeine. The availability of opioid containing analgesics for self-medication will provide parity of treatment for the population as a whole.

Accordingly, in a first aspect there is provided a self-medication process for the relief of pain including the step of administering to oneself a composition including an opioid other than codeine as a medicine for the relief of pain.

Optionally, the composition is administered at least once in an amount of up to about 10 mg of opioid per dose. It is a further option that the total amount of opioid administered is less than 500 mg. Yet a further option is that a plurality of doses is administered over a period of time up to 4 days.

Preferably, the opioid is selected from the group consisting of morphine, hydromorphone and oxycodone.

In a second aspect, the present invention provides a pharmaceutical composition for use as a self-medication for the relief of pain including an opioid other than codeine and a pharmaceutically acceptable adjuvant, diluent or excipient.

Preferably, the opioid is present in an amount of up to about 10 mg per dose. More preferably, the opioid is selected from the group consisting of morphine, hydromorphone and oxycodone.

Optionally, the pharmaceutical composition further includes a non-steroidal anti-inflammatory compound. Preferably, the non-steroidal anti-inflammatory compound is paracetamol. More preferably, if present, the paracetamol is present in an amount of up to about 500 mg per dose.

The above composition may additionally be adapted to prevent or minimise isolation of the opioid from the composition. This can be achieved by providing a further compound in the composition that has at least one characteristic that is sufficient to prevent the opioid of the composition from being isolated. Optionally, the further compound is codeine.

In a further aspect the present invention provides an over-the-counter medicine including an opioid other than codeine for providing relief from pain.

In yet another aspect the present invention provides a tablet, capsule or caplet for use as a self-medication for the relief of pain including up to about 10 mg of an opioid other than codeine, about 500 mg of paracetamol and a pharmaceutically acceptable adjuvant, diluent or excipient.

In an additional aspect of the invention there is provided a kit for providing relief from pain including:
(a) a composition as described above; and
(b) instructions for using the composition as a self-medication for the relief of pain according to the process as described above.

Preferably, the composition in the kit is formulated into a plurality of units. More preferably, each unit includes up to about 10 mg of opioid. Optionally, the kit includes a total amount of opioid of less than 500 mg.

In a further aspect the present invention provides use of an opioid other than codeine in the manufacture of a medicament for use in a process as described above.

In yet another aspect the present invention provides a process for the relief of pain including the step of administering to oneself a composition including an opioid other than codeine as a medicine for the relief of pain in accordance with a prescription.

Optionally, the composition is administered at least once in an amount of up to about 40 mg of opioid per dose. it is a further option that the total amount of opioid administered is less than 500 mg. Yet a further option is that a plurality of doses is administered over a period of time up to 4 days.

Preferably, the opioid is selected from the group consisting of morphine, hydromorphone and oxycodone.

In a further aspect, the present invention provides a pharmaceutical composition for use as a prescription medicine for the relief of pain including an opioid other than codeine and a pharmaceutically acceptable adjuvant, diluent or excipient.

Preferably, the opioid is present in an amount of up to about 40 mg per dose. More preferably, the opioid is selected from the group consisting of morphine, hydromorphone and oxycodone.

Optionally, the pharmaceutical composition further includes a non-steroidal anti-inflammatory compound. Preferably, the non-steroidal anti-inflammatory compound is paracetamol. More preferably, if present, the paracetamol is present in an amount of up to about 500 mg per dose.

The above composition may additionally be adapted to prevent or minimise isolation of the opioid from the composition. This can be achieved by providing a further compound in the composition that has at least one characteristic that is sufficient to prevent the opioid of the composition from being isolated. Optionally, the further compound is codeine.

### DETAILED DESCRIPTION OF THE EMBODIMENTS

The present invention will now be described in detail with reference to particular embodiments. While instructive, the following description is not intended to be limiting on the scope of the invention.

In a first embodiment the present invention provides a self-medication process for the relief of pain including the step of administering to oneself a composition including an opioid other than codeine as a medicine for the relief of pain.

As described herein, self-medication is the administration to oneself of a drug or medicine other than with a prescription or on medical advice. A person practices self-medication when he or she engages in self-treatment of a disease or ailment without consulting with a medical practitioner.

When self-medicating, a person will first diagnose their condition, by observing and analysing his or her symptoms, then determine how the condition should be treated, and finally administer to himself or herself the determined treatment. Typically, when an OTC medicine is obtained in order to treat the condition, the person will read the generic instructions provided with the medicine and self-administer the medicine in accordance with those instructions.

For example, when self-medicating to treat a headache, a person will observe the pain in their head and diagnose the existence of the headache. Depending on the intensity of the headache, the person may then determine that the headache needs to be treated using an analgesic in order to relieve the symptoms. A person will then obtain an analgesic and administer a dose of the analgesic to themselves in accordance with the generic instructions provided with the analgesic.

Self-medication for the relief of pain is known using many types of analgesics, such as paracetamol, ibuprofen and codeine. As discussed above, codeine as a broadly applicable analgesic is of limited use due to the existence of sub-populations that either over- or under-metabolise codeine. The metabolism problems experienced with codeine can be overcome by administering alternative opioids, such as morphine. However, opioids generally have not been legally available for self-medication, given the current views on the potential for abuse, misuse, dependence and severe adverse side effects.

Administration to oneself is generally the step of actually undertaking the determined treatment, for example, ingesting a tablet or applying a cream or ointment.

The self-medication process the subject of the present invention is for the relief of pain.

Pain includes both acute and chronic physical pain. The pain may result from a disease or condition which is transitory and/or will heal naturally, for example a shallow cut or laceration, sprain, headache, broken rib, common cold, influenza, migraine, muscular aches and period pain. Alternatively, the pain may result from a disease or condition which is itself treated or cured by self-medication, such as an abrasion or burn. The pain may result from a disease or condition which requires professional medical attention in order to be cured or alleviated, for example toothache, broken limbs or bacterial infection. Pain may also result from prior medical intervention, such as that required in order to treat a disease or condition, for example amputation, surgical incision, excision, debridement or removal of tissue.

Relief from pain encompasses reducing, decreasing, lessening, abating or alleviating pain in order to render it more tolerable or completely remove the sensation.

Opioids suitable for use in the invention include those listed in Schedule 1 of the United Nations Single Convention on Narcotic Drugs, 1961, as amended by the 1972 Protocol amending the Single Convention on Narcotic Drugs, 1961. In particular, opioids suitable for use in the present invention include any opioids currently used as medicinal analgesics, such as morphine, oxycodone, hydromorphone, fentanyl, pethidine, alfentanil and ketobemidone.

Preferably, the opioid is selected from the group consisting of morphine, oxycodone and hydromorphone.

Typically, and particularly in compositions for self-medication, which may be sold as OTC medicines, the opioid is provided in an amount of up to about 10 mg per dose. The opioid may also be provided in an amount of from about 0.1 mg to 5 mg per dose. Otherwise, the opioid is provided in an amount of from about 0.5 to 3 mg per dose.

Where the opioid is morphine, the morphine may be provided in an amount of not more than about 10 mg per dose. Also, the morphine may be provided in an amount of from about 1.5 mg to 10 mg per dose, more preferably from about 2.0 to 6 mg per dose. Otherwise, the morphine may be provided in an amount of about 1.0 to 3 mg per dose.

Where the opioid is oxycodone, the oxycodone may be provided in an amount of not more than about 10 mg per dose. Also, the oxycodone may be provided in an amount of from about 1 mg to 10 mg per dose, more preferably from about 1.5 to 5 mg per dose. Otherwise, the oxycodone is provided in an amount of about 0.5 to 2 mg per dose.

Where the opioid is hydromorphone, the hydromorphone may be provided in an amount of not more than about 7.5 mg per dose. Also, the hydromorphone may be provided in an amount of from about 0.25 mg to 7.5 mg per dose, more preferably from about 0.5 mg to 2.5 mg per dose. Otherwise, the hydromorphone may be provided in an amount of about 0.5 to 1 mg per dose.

In another embodiment, the invention provides a process for the relief of pain including the step of administering to oneself a composition including an opioid other than codeine as a medicine for the relief of pain in accordance with a prescription.

In certain embodiments, the composition includes and opioid other than codeine and oxycodone.

A prescription is generally an authorisation of a medical practitioner or like health care service provider for a medicine to be dispensed to a particular person. Typically, it is a written document or certificate signed by a medical practitioner authorising the dispensing of a medicine to that person that also includes specific directions regarding how the medicine is to be administered, such as a dosing regime. In contrast to the generic directions provided with OTC medications, which are addressed to the population at large, a prescription is given in a specific circumstance to a particular person in order to treat a specific disease or condition.

In this process, where medicines are obtained using a prescription, the opioid in the composition is typically provided in an amount of up to about 40 mg per dose. The opioid may also be provided in an amount of from about 2 mg to 15 mg per dose. Otherwise, the opioid is provided in an amount of from about 1 to 7 mg per dose.

Where the opioid is morphine, the morphine may be provided in an amount of not more than about 40 mg per dose. Also, the morphine may be provided in an amount of from about 4 mg to 40 mg per dose, more preferably from about 5.0 mg to 15 mg per dose. Otherwise, the morphine may be provided in an amount of about 6 mg per dose.

Where the opioid is oxycodone, the oxycodone may be provided in an amount of not more than about 40 mg per dose. Also, the oxycodone may be provided in an amount of from about 2.5 mg to 40 mg per dose, more preferably from about 2.5 mg to 7.5 mg per dose. Otherwise, the oxycodone is provided in an amount of about 5 mg per dose. If such amounts are used, the opioid may be combined with a non steroidal analgesic other than aspirin.

Where the opioid is hydromorphone, the hydromorphone may be provided in an amount of not more than about 15 mg per dose. Also, the hydromorphone may be provided in an amount of from about 1 mg to 15 mg per dose, more preferably from about 1.5 mg to 5 mg per dose. Otherwise, the hydromorphone may be provided in an amount of about 3 mg per dose.

In certain embodiments, there is provided a composition including an opioid other than codeine, said opioid for providing analgesia or for providing relief from pain in an individual that receives the composition, said composition being adapted for provision to an individual requiring analgesia or relief from pain as an OTC medicine or prescription medicine.

As described herein, an over-the-counter (OTC) medicine includes a pharmaceutical substance that may be legally obtained by a consumer from a pharmacy, supermarket or like outlet without presenting a prescription or other form of authorisation from a medical practitioner. In contrast, a prescription medicine includes a pharmaceutical substance that may be legally obtained by a consumer from a pharmacy or like outlet on the presentation of a prescription or other form of authorisation from a medical practitioner.

The composition can be adapted for provision as an OTC or prescription medicine in any manner known to a person skilled, in the art and commonly used for preparing OTC and prescription medicines. For example, the composition can:
(a) be packaged in a particular manner to restrict consumer access to large volumes of the active constituent, in this case the opioid, and/or to minimise the potential for inadvertently incorrectly administering the composition;
(b) further include a second active constituent that limits the amount of opioid that can safely be ingested by an individual; and/or
(c) further include a compound which limits the extractability of the opioid.

Each of these aspects of adapting the composition for provision as an OTC or prescription medicine is discussed in greater detail below.

In packaging the composition for OTC or prescription medicines, the composition is preferably adapted to form a plurality of units or doses, each unit or dose containing a selected amount of opioid. For example, the selected amount of opioid in each unit or dose is sufficient for providing analgesia or relief from acute pain for a period of 4 to 6 hours.

Typically the composition is adapted to provide a total number of units that is sufficient for providing analgesia or relief from acute pain for about 1 to 4 days. Alternatively, the composition as packaged is adapted to provide a total amount of opioid per package of 500 mg.

The composition may be adapted so as to be provided in the form of a blister pack, in which each of the plurality of units or doses is individually sealed. In one embodiment a blister pack contains about 10 to 12 doses or units.

In one embodiment, a dose is delivered by ingestion of more than one tablet, caplet, capsule or the like. That is, where a dose of opioid is, for example, 10 mg, the dose may be delivered by ingestion of two 5 mg tablets, caplets, capsules or the like.

By packaging the composition in individual units or dosages, the process of identifying what amount of composition corresponds to the desired dosage is simplified, which reduces the potential for administering an incorrect dosage inadvertently. Further, providing the composition in a blister pack provides additional means by which to monitor and determine how the composition is being administered.

In combining the opioid with a second active constituent in order to adapt it for OTC or prescription use, a second active constituent having low toxicity is added to the composition. Preferably, the second active constituent also has minimal chemical interaction with the opioid in vivo and is capable of being stably formulated with the opioid. For example, the second active constituent may be a non-steroidal analgesic, such as ibuprofen, dextropropoxyphene or paracetamol, dextromethorphan, or an antihistamine, such as fexofenadine.

The relative amounts of the opioid and the second active constituent are selected to reduce the potential for overdose of the opioid and/or severe adverse side effects from the opioid in an individual that receives the composition. This may be done by selecting an amount of the second active constituent which would mean overdose or side effects from the lower toxicity second active constituent would likely occur before overdose or adverse side effects from the opioid would be induced.

For example, if the second active constituent is paracetamol, the amount of paracetamol per unit or dose is selected to be 500 mg. Alternatively, if the second active constituent is ibuprofen, the amount of ibuprofen per unit or dose is selected to be 200 mg.

Accordingly, the following units or doses of composition are contemplated particularly for self-medication or OTC medicines:
Morphine 3 mg/dose together with paracetamol 500 mg/dose;
Oxycodone 2.0 mg/dose together with paracetamol 500 mg/dose;
Hydromorphone 0.5 mg/dose together with paracetamol 500 mg/dose.
Morphine 3 mg/dose together with ibuprofen 200 mg/dose;
Oxycodone 2.0 mg/dose together with ibuprofen 200 mg/dose;
Hydromorphone 0.5 mg/dose together with ibuprofen 200 mg/dose.

In addition, the following units or doses of composition are contemplated particularly for prescription medicines:
Morphine 7.5 mg/dose together with paracetamol 500 mg/dose;
Oxycodone 5 mg/dose together with paracetamol 500 mg/dose;
Hydromorphone 2 mg/dose together with paracetamol 500 mg/dose.
Morphine 7.5 mg/dose together with ibuprofen 200 mg/dose;
Oxycodone 5 mg/dose together with ibuprofen 200 mg/dose;
Hydromorphone 2 mg/dose together with ibuprofen 200 mg/dose.

In further embodiments, the composition is adapted for provision for self-medication or as an OTC or prescription medicine by being adapted to prevent the opioid of the composition from being isolated. This can be achieved by providing a further compound in the composition that has at least one characteristic that is sufficient to prevent or inhibit the opioid of the composition from being isolated. Typically the particular characteristic of the further compound is the same as a characteristic of the opioid. For example, the further compound may have the same solubility as the opioid.

In certain embodiments, the further compound is also an opioid. In these embodiments, the further compound is provided in an amount that is not sufficient to induce analgesia in an individual receiving the composition. Where the opioid in the composition is morphine, the further compound is preferably codeine. In this composition, the inclusion of codeine prevents the isolation of pure morphine from the composition, for example by cold water extraction. An example of a unit of this composition is as follows:
3 mg of morphine (the “opioid for providing analgesia”) together with 1 mg of codeine (the “further compound”).

In another embodiment there is provided a composition for providing analgesia or for providing relief from pain, said composition including:
an opioid other than codeine; and
a second active constituent suitable for combination with the opioid;
wherein the amount of the opioid in the composition is selected to provide for analgesia or to provide for relief from pain in an individual that receives the composition; and
the relative amounts of the opioid and second active constituent are selected to reduce the potential for overdose of the opioid and/or severe adverse side effects from the opioid in an individual that receives the composition.

A severe adverse side effect means a potentially life threatening side effect, and when used in the context of opioid administration, includes respiratory depression.

The amount of the opioid in the composition selected to provide for analgesia or to provide for relief from pain is as described above in relation to the first aspect of the invention.

The second active constituent suitable for combination with the opioid would be any active constituent having low toxicity. Preferably, the second active constituent also has minimal chemical interaction with the opioid in vivo and would be able to be stably formulated with the opioid. Such a second active constituent may be a non-steroidal analgesic (for example paracetamol or ibuprofen), dextromethorphan or an antihistamine. Preferably, the second active constituent is not kaolin.

It will be understood, in accordance with current practices, how to select the relative amounts of the opioid and second active constituent in order to reduce the potential for overdose of the opioid and/or severe adverse side effects from the opioid in an individual that receives the composition. In particular, the above discussion of the use of second active constituents to adapt the composition of the first embodiment for OTC or prescription medicines is related to this aspect.

The composition of this embodiment may further include adaptation of the composition to prevent or inhibit the opioid of the composition from being isolated, as described above. In addition, the composition of this embodiment of the invention may also be packaged so as to further adapt it for provision as an OTC medicine or prescription medicine, as described above.

The compositions of the invention can be formulated in accordance with standard pharmaceutical practice. Accordingly, the compositions may further include a pharmaceutically acceptable adjuvant, diluent or carrier. In a further aspect the present invention provides a process for the preparation of such compositions which comprises mixing a composition as described above with a pharmaceutically acceptable adjuvant, diluent or carrier.

The pharmaceutical compositions of this invention may be administered in any standard manner for administering OTC or prescription analgesic medicines, for example by oral, rectal or parenteral administration. For these purposes the compositions of this invention may be formulated by means known in the art into the form of, for example, aerosols, dry powder formulations, tablets, capsules, caplets, syrups, powders, granules, aqueous or oily solutions or suspensions, (lipid) emulsions, dispersible powders, suppositories, ointments, creams, drops and sterile injectable aqueous or oily solutions or suspensions.

Preferably, the compositions of this invention are formulated for oral administration.

In other embodiments there is provided a kit for providing relief from pain including:
(a) a composition as described above; and
(b) instructions for using the composition as a self-medication for the relief of pain according to the process as described above.

Preferably, the composition in the kit is formulated into a plurality of units. More preferably, each unit includes up to about 10 mg of opioid. Optionally, the kit includes a total amount of opioid of less than 500 mg.

In other embodiments there is provided a use of an opioid other than codeine in the manufacture of a medicament that is adapted for provision to an individual requiring analgesia or relief from pain as an OTC medicine or prescription medicine. The medicament resulting from this use may have any of the features or characteristics of the compositions described above.

Further, there is provided a use of an opioid other than codeine in the manufacture of a medicament that is adapted for provision as an OTC medicine or a prescription medicine to an individual who under-metabolises codeine requiring analgesia or relief from pain.

Further, there is provided a use of an opioid other than codeine in the manufacture of a medicament that is adapted for provision as an OTC medicine or a prescription medicine to an individual who over-metabolises codeine requiring analgesia or relief from pain.

In other embodiments there is provided a method for providing analgesia or for providing relief from acute pain to an individual, said method including providing a composition as described above to an individual requiring analgesia or having pain.

Further, there is provided a method for providing analgesia or relief from acute pain to an individual who under-metabolises codeine, said method including providing a composition as described above to an individual requiring analgesia or having pain.

Additionally, there is provided a method for providing analgesia or relief from acute pain to an individual who over-metabolises codeine, said method including providing a composition as described above to an individual requiring analgesia or having pain.

Typically the opioid is provided together with a non steroidal analgesic or paracetamol. Typically the opioid is provided as an ingestible unit or dose—i.e. a tablet, caplet, capsule or the like—having an amount of paracetamol of about 500 mg and an amount of an opioid as follows: 3 mg of morphine; or 2 mg of oxycodone; or 2 mg of hydromorphone. Typically the treatment involves administering one unit every 4 to 6 hours for a total period of no more than about 1.5 days.

In other embodiments there is provided a composition as described above when used for providing analgesia or for providing relief from pain.

In other embodiments, there is provided a use of a composition as described above for providing analgesia or for providing relief from pain.

The invention will now be described with reference to the following examples. The examples are not intended to be limiting on the scope of the invention as disclosed.

### EXAMPLE 1

### Manufacture of a Tablet Composition

A mixture of 3 mg of morphine, 500 mg of paracetamol, lactose (monohydrate) and maize starch (native) is granulated with a 5% strength solution (m/m) of polyvinylpyrrolidone in water. The granules are dried and then mixed with magnesium stearate for 5 minutes. This mixture is compressed using a conventional tablet press. A guideline force used for the compression is 15 kN.

### EXAMPLE 2

### Manufacture of a Tablet Composition

A mixture of 0.5 mg of hydromorphone, 200 mg of ibuprofen, lactose (monohydrate) and maize starch (native) is granulated with a 5% strength solution (m/m) of polyvinylpyrrolidone in water. The granules are dried and then mixed with magnesium stearate for 5 minutes. This mixture is compressed using a conventional tablet press. A guideline force used for the compression is 15 kN.

### EXAMPLE 3

### Process of Self-Medication to Relieve a Headache

An adult male observes symptoms of aching and pounding behind his eyes. He identifies the cause of the pain as a headache and determines to self-treat the headache in order to reduce or eradicate the pain. The adult male administers one tablet of example 1. The pain subsides for approximately 4 hours, at which point the adult male observes the symptoms returning. At 5 hours from the initial administration, the adult male administers a further tablet of example 1.

1. A self-medication process for the relief of pain including the step of administering to oneself a composition including an opioid other than codeine as a medicine for the relief of pain.

2. The process according to claim 1, wherein the composition is administered at least once in an amount of up to about 10 mg of opioid per dose.

3. The process according to claim 2, wherein the total amount of opioid administered is less than 500 mg.

4. The process according to claim 2, wherein a plurality of doses is administered over a period of time up to 4 days.

5. The process according to claim 1, wherein the opioid is selected from the group consisting of morphine, hydromorphone and oxycodone.

6. A pharmaceutical composition for use as a self-medication for the relief of pain including an opioid other than codeine and a pharmaceutically acceptable adjuvant, diluent or excipient.

7. The pharmaceutical composition according to claim 6 wherein the opioid is present in an amount of up to about 10 mg per dose.

8. The pharmaceutical composition according to claim 6, wherein the opioid is selected from the group consisting of morphine, hydromorphone and oxycodone.

9. The pharmaceutical composition according to claim 6, wherein the composition further includes a non-steroidal anti-inflammatory compound.

10. The pharmaceutical composition according to claim 9, wherein the compound is paracetamol.

11. The pharmaceutical composition according to claim 10, wherein the paracetamol is present in an amount of up to about 500 mg per dose.

12. The pharmaceutical composition according to claim 6, wherein the composition is adapted to prevent or minimise isolation of the opioid from the composition.

13. The pharmaceutical composition according to claim 12, wherein the composition includes codeine to prevent or minimise isolation of the opioid from the composition.

14. An over-the-counter medicine including an opioid other than codeine for providing relief from pain.

15. A tablet, capsule or caplet for use as a self-medication for the relief of pain including up to about 10 mg of an opioid other than codeine, about 500 mg of paracetamol and a pharmaceutically acceptable adjuvant, diluent or excipient.

16. A kit for providing relief from pain including:
(a) a composition according to claim 6; and
(b) instructions for using the composition as a self-medication for the relief of pain according to the process of claim 1.

17. The kit according to claim 16, wherein the composition is formulated into a plurality of units.

18. The kit according to claim 17, wherein each unit includes up to about 10 mg of opioid.

19. The kit according to claim 16, wherein the kit includes a total amount of opioid of less than 500 mg.

20. Use of an opioid other than codeine in the manufacture of a medicament for use in a process according to claim 1.

21. A process for the relief of pain including the step of administering to oneself a composition including an opioid other than codeine as a medicine for the relief of pain in accordance with a prescription.

22. The process according to claim 21, wherein the composition is administered at least once in an amount of up to about 40 mg of opioid per dose.

23. The process according to claim 22, wherein the total amount of opioid administered is less than 500 mg.

24. The process according to claim 22, wherein a plurality of doses is administered over a period of time up to 4 days.

25. The process according to claim 21, wherein the opioid is selected from the group consisting of morphine, hydromorphone and oxycodone.

26. A pharmaceutical composition for use as a prescription medicine for the relief of pain including an opioid other than codeine and a pharmaceutically acceptable adjuvant, diluent or excipient.

27. The pharmaceutical composition according to claim 26, wherein the opioid is present in an amount of up to about 40 mg per dose.

28. The pharmaceutical composition according to claim 26, wherein the opioid is selected from the group consisting of morphine, hydromorphone and oxycodone.

29. The pharmaceutical composition according to claim 26, wherein the composition further includes a non-steroidal anti-inflammatory compound.

30. The pharmaceutical composition according to claim 29, wherein the compound is paracetamol.

31. The pharmaceutical composition according to claim 30, wherein the paracetamol is present in an amount of up to about 500 mg per dose.

32. The pharmaceutical composition according to claim 26, wherein the composition is adapted to prevent or minimise isolation of the opioid from the composition.

33. The pharmaceutical composition according to claim 32, wherein the composition includes codeine to prevent or minimise isolation of the opioid from the composition.
